(19) United States
(12) Patent Application Publication
(10) Pub. No.: US 2017/0043159 A1
(43) Pub. Date: Feb. 16, 2017

(54) CLOSED-LOOP STIMULATING APPARATUS AND STIMULATING METHOD THEREOF

(71) Applicant: Winbond Electronics Corp., Taichung City (TW)

(72) Inventor: Chen-Yi Lee, Hsinchu City (TW)

(21) Appl. No.: 14/857,822

(22) Filed: Sep. 18, 2015

(30) Foreign Application Priority Data
Aug. 10, 2015 (CN) 201510486280.6

(51) Int. Cl. A61N 1/08; A61B 5/16; A61B 5/0402; A61N 1/36; A61B 5/00

(52) U.S. Cl. CPC A61N 1/08; A61N 1/36014; A61B 5/4836; A61B 5/0402; A61B 5/165

(57) ABSTRACT

Provided are a closed-loop stimulating apparatus and a stimulating method thereof, where the method includes: determining a physiological status variation of a user receiving a stimulating signal according to an electrocardiogram signal of the user, and generating the stimulating signal according to a variation of the electrocardiogram signal relative to electrocardiogram history data of the user.

### CROSS-REFERENCE TO RELATED APPLICATION

This application claims the priority benefit of Chinese application serial no. 201510486280.6, filed on Aug. 10, 2015. The entirety of the above-mentioned patent application is hereby incorporated by reference herein and made a part of this specification.

### BACKGROUND OF THE INVENTION

Field of the Invention

The invention relates a stimulating apparatus, and more particularly, to a closed-loop stimulating apparatus and a stimulating method thereof.

Description of Related Art

Tran-cutaneous Electrical Never Stimulation (TENS) achieves effects of relieving pain or enhancing blood circulation by outputting a pulse current by using low frequency. In general, the TENS stimulator is capable of sending a low voltage electrical pulse with different frequencies and intensities to a contact surface of skin at acupuncture points or places that require relieving pain or enhancing blood circulation.

The conventional electrical stimulation technique often requires use of past experiences of the user to subjectively determine whether the physiological status of a target receiving the electrical stimulation has accomplished a relieved effect during the electrical stimulation. However, whether locations, intensities and frequencies of the electrical stimulation are appropriate cannot be accurately learned by using said method since each person may have different subjective feelings.

### SUMMARY OF THE INVENTION

The invention is directed to a closed-loop stimulating apparatus and a stimulating method thereof, which are capable of accurately learning the physiological status variation of the user receiving the stimulating signal.

A closed-loop stimulating apparatus of the invention includes an electrode unit, a stimulating unit, an electrocardiogram signal generation unit, a storage unit and a control unit. The electrode unit is attached to a skin surface of a user to receive an electrical signal from the skin surface of the user. The stimulating unit is coupled to the electrode unit, and outputs a stimulating signal through the electrode unit. The electrocardiogram signal generation unit is coupled to the electrode unit, and converts the electrical signal to generate an electrocardiogram signal. The storage unit is coupled to the electrocardiogram signal generation unit, and stores electrocardiogram history data of the user. The control unit is coupled to the electrocardiogram signal generation unit, the storage unit and the stimulating unit, and controls the stimulating unit to generate the stimulating signal according to a variation of the electrocardiogram signal relative to the electrocardiogram history data.

In an embodiment of the invention, the electrocardiogram history data includes electrocardiogram signal data within a preset period started when receiving the electrical signal through the electrode unit.

In an embodiment of the invention, the storage unit further stores biological feature data of the user, and the control unit further controls the stimulating unit to generate the stimulating signal according to the biological feature data.

In an embodiment of the invention, the control unit further controls the stimulating unit to adjust a frequency and an intensity of the stimulating signal.

In an embodiment of the invention, the storage unit stores a plurality of stimulating modes corresponding to a plurality of preset mental statuses, and the control unit further determines the preset mental status conesponding to the user according to the electrocardiogram signal and controls the stimulating unit to generate the stimulating signal according to the selected stimulating mode corresponding to the preset mental status of the user.

A stimulating method of closed-loop stimulating apparatus of the invention includes the following steps. An electrical signal is received from a skin surface of a user through an electrode unit. The electrical signal is converted to generate an electrocardiogram signal. The stimulating signal is generated according to the electrocardiogram signal and electrocardiogram history data of the user.

In an embodiment of the invention, the electrocardiogram history data includes electrocardiogram signal data within a preset period started when receiving the electrical signal through the electrode unit.

In an embodiment of the invention, said stimulating method of closed-loop stimulating apparatus further includes: generating the stimulating signal according to biological feature data of the user.

In an embodiment of the invention, the step of generating the stimulating signal in said stimulating method of closed-loop stimulating apparatus includes: adjusting a frequency and an intensity of the stimulating signal.

In an embodiment of the invention, said stimulating method of closed-loop stimulating apparatus further includes the following steps. A mental status of the user is determined according to the electrocardiogram signal. The stimulating signal is generated by selecting a corresponding stimulating mode according to the mental status of the user.

Based on the above, according to the embodiments of the invention, the physiological status variation of the user receiving the stimulating signal is determined according to the electrocardiogram signal of the user, and the stimulating signal is generated according to the variation of the electrocardiogram signal relative to electrocardiogram history data. As a result, the stimulating signal may be accurately generated according to the physiological status variation of the user.

To make the above features and advantages of the present disclosure more comprehensible, several embodiments accompanied with drawings are described in detail as follows.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic diagram of a closed-loop stimulating apparatus according to an embodiment of the invention.

FIG. 2 is a schematic diagram of a stimulating method of closed-loop stimulating apparatus according to an embodiment of the invention.

FIG. 3 is a schematic diagram of a stimulating method of closed-loop stimulating apparatus according to another embodiment of the invention.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

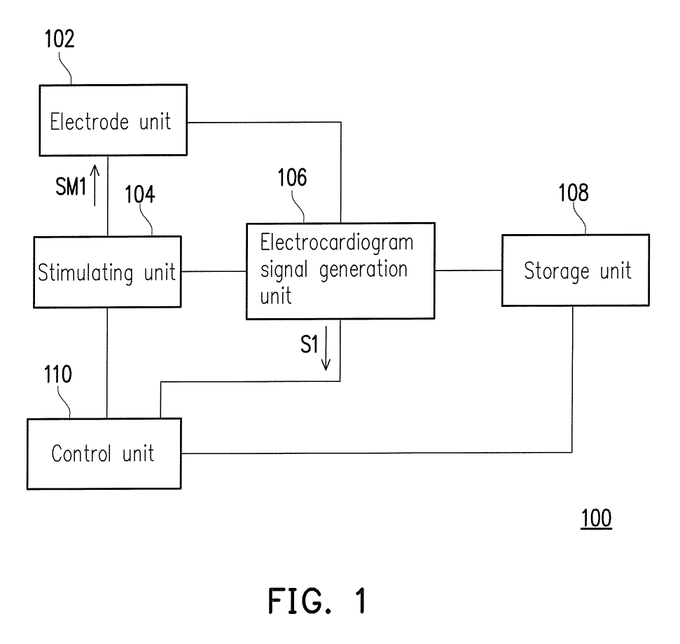

Referring to FIG. 1, FIG. 1 is a schematic diagram of a closed-loop stimulating apparatus according to an embodiment of the invention. A closed-loop stimulating apparatus 100 includes an electrode unit 102, a stimulating unit 104, an electrocardiogram signal generation unit 106, a storage unit 108 and a control unit 110.

Herein, the stimulating unit 104 is coupled to the electrode unit 102; the electrocardiogram signal generation unit 106 is coupled to the electrode unit 102 and the storage unit 108; and the control unit 110 is coupled to the stimulating unit 104, the electrocardiogram signal generation unit 106 and the storage unit 108. The electrode unit 102 may include, for example, two electrode patches attached to a skin surface of a user to receive an electrical signal from the skin surface. The stimulating unit 104 can output a stimulating signal SM1 through the electrode unit 102 to perform stimulation for the user. The stimulating signal SM1 may be, for example, a current pulse signal. The electrocardiogram signal generation unit 106 can convert the electrical signal into an electrocardiogram signal S1. The storage unit 108 can store electrocardiogram history data of the user of the closed-loop stimulating apparatus 100 (i.e., the electrocardiogram history data of the user receiving the stimulating signal SM1). The control unit 110 can control the stimulating unit 104 to generate the stimulating signal SM1 according to a variation of the electrocardiogram signal S1 relative to the electrocardiogram history data, so as to accurately generate the stimulating signal according to a physiological status variation of the user.

Herein, the electrocardiogram history data may be, for example, electrocardiogram signal data of the user stored by the storage unit 108 when the last time (or even earlier) the closed-loop stimulating apparatus 100 is used by the user (e.g., data regarding an intensity and a frequency of the stimulating signal SM1 and the corresponding electrocardiogram signal S1 previously stored when the closed-loop stimulating apparatus 100 is used by the user in the past). Alternatively, the storage unit 108 can also store the electrocardiogram signal data within a preset period started when receiving the electrical signal through the electrode unit 102 since the first time the user uses the closed-loop stimulating apparatus 100, and use said electrocardiogram signal data as the electrocardiogram history data. Accordingly, by controlling the stimulating unit 104 to generate the stimulating signal SM1 according to the variation of the electrocardiogram signal S1 relative to the electrocardiogram history data, the corresponding stimulating signal SM1 may be provided according to different physiological conditions and physiological statuses of the users, so as to accomplish the effect of customizing the closed-loop stimulating apparatus 100. For example, a stimulating signal M1 having the frequency and the intensity which are suitable for the user may be provided according to the variation of the electrocardiogram signal S1 relative to the electrocardiogram history data, so as improve the efficiency of stimulating the user while preventing the stimulating signal M1 from having excessively weak or strong intensity or excessively high or low frequency.

In some embodiments, the storage unit 108 may further store biological feature data of the user, such as height, weight, age, gender, body fat ratio, medical history of the user and so on. Other than controlling the stimulating unit 104 to generate the stimulating signal SM1 according to the electrocardiogram history data, the control unit 110 can also control the stimulating unit 104 to generate the stimulating signal SM1 according to biological feature data in order to generate the stimulating signal SM1 that is more suitable for the user.

In addition, the storage unit 108 may also store a plurality stimulating modes corresponding to a plurality of preset mental statuses. The preset mental statuses may include, for example, excitement, tension, relaxation, depression, irritability, stability, and so on. The control unit 110 can analyze activity of the sympathetic nerve and the parasympathetic nerve according to the electrocardiogram signal S1 to determine a current mental status of the user and thereby select the corresponding stimulating mode to control the stimulating unit 104 for outputting the stimulating signal SM1. By analyzing activity of the sympathetic nerve and the parasympathetic nerve according to the electrocardiogram signal S1 to determine the current mental status of the user, instead of control by subjective judgment, the user can clearly handle influences from the stimulating signal SM1 to the physiological conditions of the user. By providing the stimulating signal SM1 according to the mental status of the user, the stimulating signal SM1 that is suitable for the user may be provided, so that the closed-loop stimulating apparatus 100 can be used more efficiently. It should be noted that, besides the usage in determination of the mental status of the user, the electrocardiogram signal S1 may also include various physiological condition information of the user (e.g., heartbeat frequency, heartbeat intensity, heart rate variability), which can be all used as a basis for adjusting the stimulating signal SM1 in addition to the mental status of the user described above.

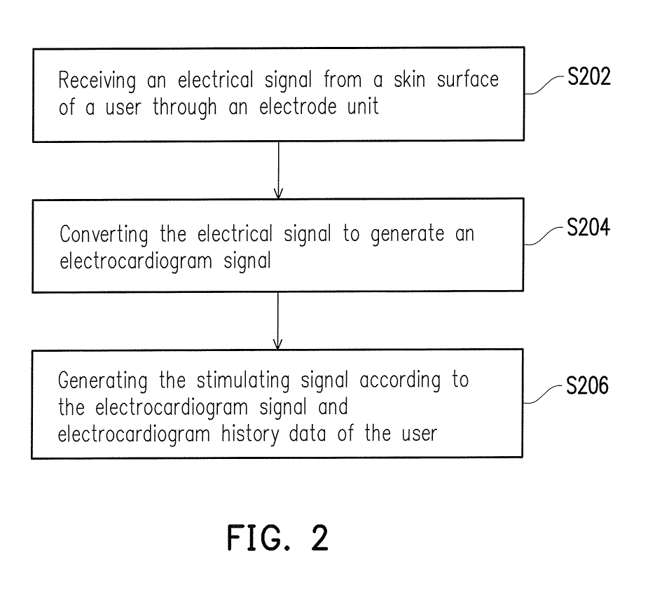

Referring to FIG. 2, FIG. 2 is a schematic diagram of a stimulating method of closed-loop stimulating apparatus according to an embodiment of the invention. In view of the foregoing embodiments, it can be known that the stimulating method of closed-loop stimulating apparatus can include the following steps. First of all, an electrical signal is received from a skin surface of a user through an electrode unit (step S202), wherein the electrode unit can include, for example, two electrode patches. Subsequently, the electrical signal is converted to generate an electrocardiogram signal (step S204). Then, the stimulating signal is generated according to a variation of the electrocardiogram signal relative to electrocardiogram history data of the user (step S206). For example, the frequency and the intensity of the stimulating signal may be adjusted according to the variation of the electrocardiogram signal relative to electrocardiogram history data. Herein, the electrocardiogram history data may be, for example, electrocardiogram signal data stored when the last time (or even earlier) the closed-loop stimulating apparatus 100 is used by the user, and the electrocardiogram signal may be, for example, the intensity and the frequency of the stimulating signal and the corresponding electrocardiogram signal stored when the closed-loop stimulating apparatus is used by the user in the past. Alternatively, the electrocardiogram history data may also be, for example, the electrocardiogram signal data within a preset period started when receiving the electrical signal through the electrode unit since the first time the user uses the closed-loop stimulating apparatus. Further, in some embodiments, the stimulating signal may further be generated according to biological feature data of the user (e.g., height, weight, age, gender, body fat ratio, medical history of the user and so on) in step S206, so as to generate the stimulating signal that is more suitable for the user.

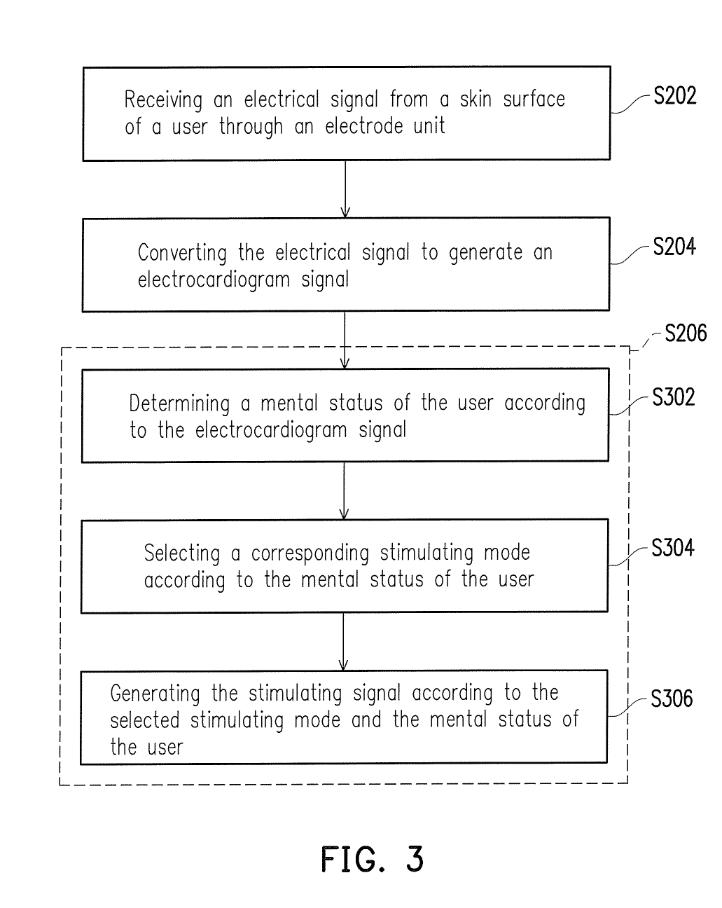

Referring to FIG. 3, FIG. 3 is a schematic diagram of a stimulating method of closed-loop stimulating apparatus according to another embodiment of the invention. In the present embodiment, after step S204 is performed, a mental status of the user is determined according to the electrocardiogram signal (step S302). Then, a corresponding stimulating mode is selected according to the mental status of the user (step S304). Lastly, the stimulating signal is generated according to the selected stimulating mode and the mental status of the user (step S306). Accordingly, the efficiency of using the closed-loop stimulating apparatus may be improved by generating the stimulating signal according to the variation of the electrocardiogram signal relative to electrocardiogram history data and the mental status of the user.

In summary, according to the embodiments of the invention, the physiological status variation of the user receiving the stimulating signal is determined according to the electrocardiogram signal of the user, and the stimulating signal is generated according to the variation of the electrocardiogram signal relative to electrocardiogram history data. As a result, the stimulating signal may be accurately generated according to the physiological status variation of the user. In some embodiments, the efficiency of using the closed-loop stimulating apparatus may be further improved by generating the stimulating signal according to the mental status of the user.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. A closed-loop stimulating apparatus, comprising:
an electrode unit, attached to a skin surface of a user to receive an electrical signal from the skin surface of the user;
a stimulating unit, coupled to the electrode unit, and outputting a stimulating signal through the electrode unit;
an electrocardiogram signal generation unit, coupled to the electrode unit, and converting the electrical signal to generate an electrocardiogram signal;
a storage unit, coupled to the electrocardiogram signal generation unit, and storing electrocardiogram history data of the user; and
a control unit, coupled to the electrocardiogram signal generation unit, the storage unit and the stimulating unit, and controlling the stimulating unit to generate the stimulating signal and to adjust a frequency and an intensity of the stimulating signal according to a variation of the electrocardiogram signal relative to the electrocardiogram history data.

2. The closed-loop stimulating apparatus of claim 1, wherein the electrocardiogram history data comprises electrocardiogram signal data within a preset period started when receiving the electrical signal through the electrode unit.

3. The closed-loop stimulating apparatus of claim 1, wherein the storage unit further stores biological feature data of the user, and the control unit further controls the stimulating unit to generate the stimulating signal according to the biological feature data.

4. (canceled)

5. The closed-loop stimulating apparatus of claim 1, wherein the storage unit stores a plurality of stimulating modes corresponding to a plurality of preset mental statuses, and the control unit further determines the preset mental status corresponding to the user according to the electrocardiogram signal and controls the stimulating unit to generate the stimulating signal according to the selected stimulating mode corresponding to the preset mental status of the user.

6. A stimulating method of closed-loop stimulating apparatus, comprising:
receiving, through an electrode unit of the close-loop stimulating apparatus, an electrical signal from a skin surface of a user;
converting, by an electrocardiogram signal generation unit of the close-loop stimulating apparatus, the electrical signal to generate an electrocardiogram signal; and
generating, by a stimulating unit of the close-loop stimulating apparatus, a stimulating signal and adjusting a frequency and an intensity of the stimulating signal according to a variation of the electrocardiogram signal relative to electrocardiogram history data of the user.

7. The stimulating method of closed-loop stimulating apparatus of claim 6, wherein the electrocardiogram history data comprises electrocardiogram signal data within a preset period started when receiving the electrical signal through the electrode unit.

8. The stimulating method of closed-loop stimulating apparatus of claim 6, further comprising:
generating, by the stimulating unit, the stimulating signal according to biological feature data of the user.

9. (canceled)

10. The stimulating method of closed-loop stimulating apparatus of claim 6, further comprising:
determining, by a control unit of the close-loop stimulating apparatus, a mental status of the user according to the electrocardiogram signal; and
generating, by the stimulating unit, the stimulating signal by selecting a corresponding stimulating mode according to the mental status of the user.
